(19) United States
(12) Patent Application Publication
(10) Pub. No.: US 2010/0069400 A1
(43) Pub. Date: Mar. 18, 2010

(54) METHODS FOR TREATING INFLAMMATION AND RELATED CONDITIONS

(75) Inventors: Scott Malstrom, Reading, MA (US); Hellen Mulomi Amuguni, Worcester, MA (US); Clary Clish, Reading, MA (US); Amy Anderson Deik, Beverly, MA (US); David White, Norwell, MA (US)

Correspondence Address: HARNESS, DICKEY, & PIERCE, P.L.C, 7700 Bonhomme, Suite 400, ST. LOUIS, MO, 63105, US

(73) Assignee: Ore Pharmaceuticals Inc., Gaithersburg, MD (US)

(21) Appl. No.: 12/500,761

(22) Filed: Jul. 10, 2009

Related U.S. Application Data
(60) Provisional application No. 61/079,808, filed on Jul. 10, 2008.

(51) Int. Cl. A61K 31/496; C12N 5/00; A61P 29/00

(52) U.S. Cl. 514/254.05; 435/375

(57) ABSTRACT

A method for treating an inflammatory condition or immune disorder comprises administering to a subject having such a condition or disorder a therapeutically effective amount of at least one compound selected from irindalone, physiologically active enantiomers thereof, and pharmaceutically acceptable salts thereof. The invention further provides a method for elevating levels of anti-inflammatory cytokines such as IL-10 and IL-13 while inhibiting expression of pro-inflammatory cytokines. A pharmaceutical composition, useful for example in topical treatment of psoriasis, comprises a therapeutically effective amount of at least one compound selected from irindalone, physiologically active enantiomers thereof, and pharmaceutically acceptable salts thereof, in a vehicle comprising at least one pharmaceutically acceptable excipient, the vehicle being adapted for topical administration to skin of a subject.

This application claims the benefit of U.S. provisional application Ser. No. 61/079,808 filed on Jul. 10, 2008, the disclosure of which is incorporated by reference herein in its entirety.

### FIELD OF THE INVENTION

The present invention relates to therapeutic methods for treating inflammatory conditions and immunological disorders, and to methods for modulating production or activity of transcription factors, and of cytokines regulated thereby, involved in mediating inflammatory and immune responses.

### BACKGROUND

Inflammation is a necessary biological response to harmful stimuli such as wounding and infection, and is subject to a complex of regulatory processes in the body, involving the immune system and other biochemical mechanisms. In a very large and varied group of diseases, regulation of inflammatory and immune response can be disturbed, leading to unchecked inflammation that can seriously impair the normal functioning of affected tissues and organs.

### IL-10 and IL-13

Interleukin 10 (IL-10) is a multifunctional cytokine that inhibits inflammatory responses in a wide variety of cell types. See, for example, the review article by Moore et al. (2001) Annu. Rev. Immunol. 19:683-765. Some of the anti-inflammatory actions of IL-10 appear to be related to inhibition of NF-κB; however, it has also been found that IL-10 inhibits transcription of IL-5, which is independent of NF-κB. IL-10 is reported to inhibit synthesis of IL-1β, TNF-α, IL-6, IL-4, IL-5, monocyte inflammatory protein 1α (MIP-1α), CCL5, IL-8 and eotaxin; it also inhibits expression of pro-inflammatory enzymes such as inducible nitric oxide synthase (iNOS) and cyclooxygenase 2 (COX-2).

Ilodecakin, a human recombinant IL-10, has been investigated as a therapeutic in various diseases including cancer, diabetes, inflammatory bowel disease (IBD), rheumatoid arthritis and GVHD, and has recently been under development for reduction of scarring in skin.

Li & He (2004) World J. Gastroenterol. 10:620-625 have reviewed IL-10-directed therapies in IBD, including administration of recombinant IL-10, gelatin microspheres containing IL-10 or an IL-10 bacterium, Lactococcus lactis.

Friedrich et al. (2002) J. Invest. Dermatol. 118:672-677 reported results of treatment of psoriasis with recombinant IL-10 in a placebo-controlled study. Incidence of relapse was reportedly decreased and relapse-free interval prolonged by treatment with IL-10.

IL-13 is another member of the interleukin cytokine family that, like IL-10, has anti-inflammatory properties.

As an alternative to administration of exogenous IL-10, administration of a compound that increases endogenous cellular levels of IL-10 or IL-13 would represent an important advance in treatment of inflammatory and immunological disorders.

### Transcription Factors AP-1 and NF-κB

The transcription factors activator protein 1 (AP-1) and nuclear factor kappa-B (NF-κB) are involved in regulating expression of a number of genes involved in mediating inflammatory and immune responses, and accordingly play key roles in initiation and perpetuation of inflammatory and immunological disorders. See, for example, the review articles individually cited below.

Baldwin (2001) J. Clin. Invest. 107:241-246.

Firestein & Manning (1999) Arthritis and Rheumatism 42:609-621.

Peitz (1997) Curr. Opin. Biotech. 8:467-473.

AP-1 regulates transcription of genes including those involved in production of pro-inflammatory cytokines including TNF-α and interleukins 1 and 2 (IL-1 and IL-2), as well as matrix metalloproteases. NF-κB regulates transcription of genes including those involved in production of TNF-α, IL-1, IL-2 and IL-6, adhesion molecules such as E-selectin, and chemokines such as CCL5 (formerly known as RANTES) and CXCL1 (formerly known as GRO1 or KC) among others. Drug therapies targeting TNF-α, expression of which is regulated by both NF-κB and AP-1, have been shown to be highly efficacious in several inflammatory human diseases including rheumatoid arthritis and Crohn's disease.

### SUMMARY OF THE INVENTION

It has now been discovered that irindalone, a compound previously known as a peripherally acting serotonin (5-hydroxytryptamine) receptor 2A (5-HT_{2A}) antagonist, is a modulator of activity of AP-1 and NF-κB, and of production of pro-inflammatory cytokines. Of particular interest is the discovery that irindalone increases levels of the anti-inflammatory cytokines IL-10 and IL-13.

Irindalone (also known as Lu 21-098, GL10002 or ORE10002) can be described by the chemical name (+)-(1R,3S)-1-[2-[4-[3-(p-fluorophenyl)-1-indanyl]-1-piperazinyl]-ethyl]-2-imidazolidinone or 1-(2-(4-((1R,3S)-3-(4-fluorophenyl)-2,3-dihydro-1H-inden-1-yl)piperazin-1-yl)ethyl)imidazolidin-2-one, and has the following structure:

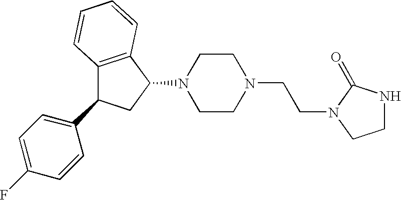

Irindalone has relatively strong affinity (IC₅₀ of 3.4 nM) for 5-HT_{2A} and somewhat weaker affinity for adrenergic receptor al and histamine H₁ receptor. The (−)-enantiomer (Lu 21-099) has weaker 5-HT_{2A} affinity. See, for example, Hyttel et al. (2004) Drug Devel. Res. 15:389-404. Irindalone was formerly of interest for potential treatment of hypertension, and has more recently been tested as an antidepressant. Irindalone has been proposed as a component of various combination therapies, but has never been brought to market.

In one embodiment, the invention provides a method for treating an inflammatory condition, comprising administering to a subject having such a condition a therapeutically effective amount of at least one compound selected from irindalone, physiologically active enantiomers thereof, and pharmaceutically acceptable salts thereof.

In some aspects, the inflammatory condition is a skin condition such as psoriasis; for such use the compound can be administered topically.

A pharmaceutical composition comprising a therapeutically effective amount of at least one compound selected from irindalone, physiologically active enantiomers thereof, and pharmaceutically acceptable salts thereof, in a vehicle comprising at least one pharmaceutically acceptable excipient, the vehicle being adapted for topical administration to skin of a subject, represents a further embodiment of the invention.

Another embodiment of the invention provides a method for treating a disorder responsive to increased cellular level of one or more anti-inflammatory cytokines such as IL-10 and/or IL-13, comprising administering to a subject having such a disorder a therapeutically effective amount of at least one compound selected from irindalone, physiologically active enantiomers thereof, and pharmaceutically acceptable salts thereof.

Yet another embodiment of the invention provides a method for treating a disorder related to increased or excessive activity of one or more pro-inflammatory transcription factors such as AP-1 and/or NF-κB and/or increased or excessive production of one or more pro-inflammatory cytokines such as TNF-α, IL-1α, CXCL1 or eotaxin, comprising administering to a subject having such a disorder a therapeutically effective amount of at least one compound selected from irindalone, physiologically active enantiomers thereof, and pharmaceutically acceptable salts thereof.

Yet another embodiment of the invention provides a method for elevating level of at least one cytokine selected from the group consisting of IL-10 and IL-13 in a cell, comprising contacting the cell with at least one compound selected from irindalone, physiologically active enantiomers thereof, and pharmaceutically acceptable salts thereof, in an amount effective to elevate level of the at least one cytokine.

Yet another embodiment of the invention provides a method for inhibiting production of at least one cytokine selected from the group consisting of TNF-α, IL- 1α, CXCL1 and eotaxin in a cell, comprising contacting the cell with at least one compound selected from irindalone, physiologically active enantiomers thereof, and pharmaceutically acceptable salts thereof, in an amount effective to inhibit production of the at least one cytokine.

Yet another embodiment of the invention provides a method for treating or preventing an immune disorder in a subject, comprising administering to the subject a therapeutically effective amount of at least one compound selected from irindalone, physiologically active enantiomers thereof, and pharmaceutically acceptable salts thereof.

Other embodiments, including particular aspects of the embodiments summarized above, will be evident from the detailed description that follows.

### BRIEF DESCRIPTION OF THE DRAWINGS

FIG. 1 shows (A) in vivo imaging of modulation by irindalone of LPS-induced NF-κB activation using NF-κB:LUC transgenic mice; and (B) a plot of fold change in NF-κB activation in various body regions of the mice at 2, 4 and 6 hours after LPS delivery.

FIG. 2 shows a plot of fold change in NF-κB activation derived from in vivo imaging results of modulation by irindalone of LPS-induced NF-κB activation using NF-κB:LUC transgenic mice, at 2, 4, 6 and 24 hours after LPS delivery.

FIG. 3 shows plots of effect of irindalone on (A) LPS-induced eotaxin cytokine levels; (B) LPS-induced TNF-α cytokine levels; (C) LPS-induced IL-10 cytokine levels; and (D) LPS-induced IL-1α cytokine levels; all in mouse plasma at 2, 4, 6 and 24 hours.

FIG. 4 shows a plot of modulation by irindalone of LPS-induced NF-κB activation in organs and tissues harvested from NF-κB:LUC transgenic mice. Results, based on units of light/μg protein, are normalized to saline control.

FIG. 5 shows (A) in vivo imaging results of modulation by dexamethasone or irindalone on TPA-induced AP-1 activation using AP-1:LUC transgenic mice; and (B) a plot of fold change in AP-1 activation.

FIG. 6 shows a plot of effect of irindalone on CdCl₂-induced HO-1 promoter in HO-1:LUC transgenic mice at 4, 6 and 24 hrs after CdCl₂ exposure.

FIG. 7 shows a plot of effect of irindalone on plasma levels of CXCL1 chemokine (GRO/KC) and IL-13 cytokine in untreated rats.

### DETAILED DESCRIPTION

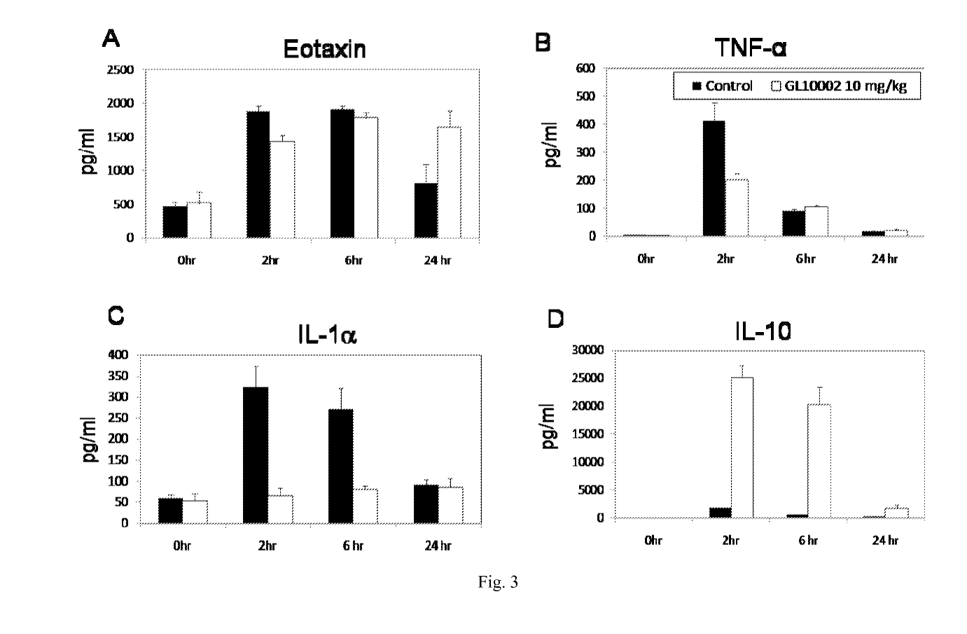

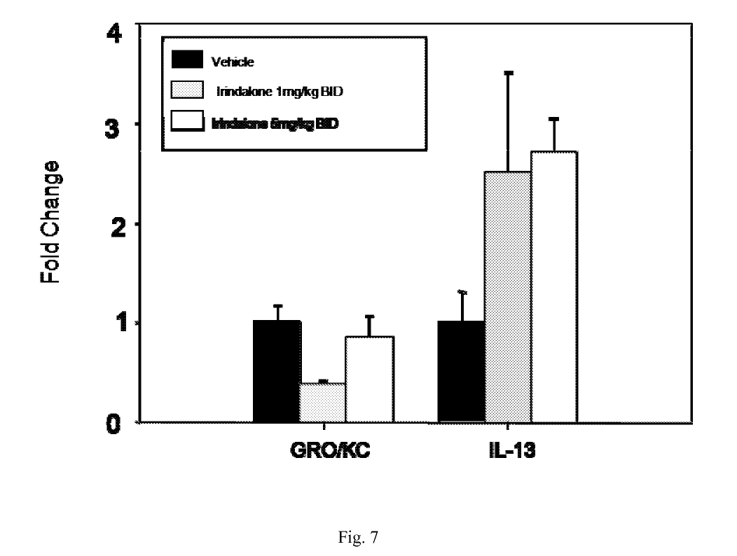

A key discovery underlying the present invention is that administration of irindalone in an in vivo murine inflammation model results in a substantial and sustained increase in endogenous levels of the anti-inflammatory cytokine IL-10 (see Example 2 and FIG. 3(D)). A similar result has been obtained in the case of another anti-inflammatory cytokine, IL-13 (see Example 6 and FIG. 7). Concomitant reductions in NF-κB activity and in levels of pro-inflammatory cytokines such as TNF-α, IL-1α and eotaxin were observed for a short period after irindalone administration, but at later time periods some of these reductions, particularly the reduction in NF-κB activity, were nullified or even reversed. Without being bound by theory, it is thought that the resurgence in NF-κB activity after an initial suppression may reflect a natural tendency for balance of pro- and anti-inflammatory factors to be restored following stimulation of IL-10 and/or IL-13 production. Nonetheless, it is believed that the dramatic increase in IL-10 and IL-13 resulting from irindalone administration indicates a potent but previously unknown anti-inflammatory effect of this drug.

Accordingly, the present invention provides, in some embodiments, a method for treating an inflammatory condition, comprising administering to a subject having such a condition a therapeutically effective amount of at least one compound selected from irindalone, physiologically active enantiomers thereof, and pharmaceutically acceptable salts thereof.

The subject herein can be any species of animal, more particularly a mammalian species including, but not limited to, primates (e.g., human subjects), household pets including dogs and cats, livestock including cattle, sheep, goats and horses, animals used as therapeutic models such as rabbits, rats and mice, and the like. The present disclosure is primarily but not exclusively directed to embodiments wherein the subject is human.

A wide variety of inflammatory conditions are treatable by a method of the invention. As used herein, the term “inflammatory condition” refers to a condition or disorder associated with one or more aberrant physiological processes or other physiological responses (such as responses to an injurious or noxious stimulus) that result in a pathophysiological state of inflammation. An inflammatory condition can be either acute or chronic, and can result from infection or from a non-infectious cause. Inflammatory conditions having infectious causes include meningitis, encephalitis, uveitis, colitis, tuberculosis, dermatitis and adult respiratory distress syndrome. Non-infectious causes of inflammatory conditions include trauma (burns, cuts, contusions, crush injuries, etc.), autoimmune diseases, and organ rejection episodes.

Thus, inflammatory conditions, or conditions having an inflammatory component, that can be treated by a method of the invention include
atherosclerosis (arteriosclerosis);
autoimmune conditions such as multiple sclerosis, systemic lupus erythematosus, polymyalgia rheumatica (PMR), gouty arthritis, degenerative arthritis, tendonitis, bursitis, psoriasis, fibrosis, arthrosteitis, rheumatoid arthritis and other forms of inflammatory arthritis, Sjögren's syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroiditis, Graves' disease, Goodpasture's disease, mixed connective tissue disease, sclerosing cholangitis, inflammatory bowel disease (IBD) including Crohn's Disease (regional enteritis) and ulcerative colitis, pernicious anemia, and inflammatory dermatoses;
usual interstitial pneumonitis (UIP), asbestosis, silicosis, bronchiectasis, berylliosis, talcosis, all forms of pneumoconiosis, sarcoidosis (in the lung and in any other organ), desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa);
sepsis;
inflammatory dermatoses not presumed to be autoimmune;
chronic active hepatitis;
delayed-type hypersensitivity reactions (e.g., poison ivy deiivatitis);
pneumonia or other respiratory tract inflammation due to any cause;
adult respiratory distress syndrome (ARDS) from any etiology;
encephalitis with inflammatory edema;
immediate hypersensitivity reactions including, but not limited to, asthma, hayfever, cutaneous allergies, and acute anaphylaxis;
diseases involving acute deposition of immune complexes, including, but not limited to, rheumatic fever, acute and/or chronic glomerulonephritis due to any etiology, including specifically post-infectious (e.g., post-streptococcal) glomerulonephritis, and acute exacerbations of systemic lupus erythematosus;
pyelonephritis;
cellulitis;
cystitis;
acute and/or chronic cholecystitis;
conditions producing transient ischemia anywhere along the gastrointestinal tract, bladder, heart, or other organ, especially those prone to rupture;
sequelae of organ transplantation or tissue allograft, including allograft rejection in the acute time period following allogeneic organ or tissue transplantation and chronic host-versus-graft rejection;
   and combinations thereof.

The term “inflammatory condition” also includes appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, tonsillitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, hepatitis, necrotizing enterocolitis and combinations thereof.

In certain aspects, the inflammatory condition treatable by a method of the invention is or includes inflammation of the skin, including but not limited to psoriasis, eczema, rosacea, acne, burns, dermatitis and ultraviolet radiation damage including sunburn.

In other particular aspects, the inflammatory condition treatable by a method of the invention is or includes IBD, more particularly Crohn's disease or ulcerative colitis; sepsis; arthritis; multiple sclerosis or a combination thereof.

In some embodiments, a method is provided for treating a disorder responsive to increased cellular level of one or more anti-inflammatory cytokines, comprising administering to a subject having such a disorder a therapeutically effective amount of at least one compound selected from irindalone, physiologically active enantiomers thereof, and pharmaceutically acceptable salts thereof.

A “disorder responsive to increased cellular level of one or more anti-inflammatory cytokines” herein is any disorder that can be ameliorated by increased amounts of anti-inflammatory cytokines such as IL-10 and/or IL-13 in cells or tissues of a subject, whether such cytokines are produced endogenously or supplied exogenously. Such disorders can be, but are not necessarily, associated with insufficient levels of IL-10 or IL-13; indeed in some embodiments the levels of IL-10 and/or IL-13 are within normal ranges, yet increasing production of one or both of these cytokines can ameliorate the condition.

Examples of such disorders include, without limitation, endocrine disorders, rheumatic disorders, collagen diseases, dermatologic diseases, allergic diseases, ophthalmic diseases, respiratory diseases, hematologic diseases, gastrointestinal diseases, inflammatory diseases, autoimmune diseases, diabetes, obesity, neoplastic diseases and combinations thereof. Such disorders can also include cancer and tumor disorders, such as solid tumors, lymphomas and leukemia; and fungal infections such as mycosis fungoides. In some aspects, the disorder can be any condition, disease or disorder that has an inflammatory or immune component, including, but not limited to, transplant rejection (e.g., kidney, liver, heart, lung, pancreas (e.g., islet cells), bone marrow, cornea, small bowel, skin allografts, skin homografts (such as employed in burn treatment), heart valve xenografts, serum sickness and GVHD); autoimmune diseases such as rheumatoid arthritis, psoriatic arthritis, multiple sclerosis, Type I and Type II diabetes, juvenile diabetes, obesity, asthma, IBD (such as Crohn's disease and ulcerative colitis), pyoderma gangrenum, lupus (systemic lupus erythematosis), myasthenia gravis, psoriasis, dermatitis, dermatomyositis, eczema, seborrhoea, pulmonary inflammation, uveitis, hepatitis, Graves' disease, Hashimoto's thyroiditis, autoimmune thyroiditis, Behcet's or Sjögren's syndrome (dry eyes/mouth), pernicious or immunohemolytic anemia, atherosclerosis, Addison's disease (autoimmune disease of the adrenal glands), idiopathic adrenal insufficiency, autoimmune polyglandular disease (autoimmune polyglandular syndrome), glomerulonephritis, scleroderma, morphea, lichen planus, vitiligo (depigmentation of the skin), alopecia areata, autoimmune alopecia, autoimmune hypopituitarism, Guillain-Barré syndrome and alveolitis; T-cell-mediated hypersensitivity diseases, including contact hypersensitivity, delayed-type hypersensitivity, contact dermatitis (including that due to poison ivy), urticaria, skin allergies, respiratory allergies (including hayfever and allergic rhinitis) and gluten-sensitive enteropathy (celiac disease); inflammatory diseases such as osteoarthritis, acute pancreatitis, chronic pancreatitis, acute respiratory distress syndrome, Sézary syndrome and vascular diseases which have an inflammatory and/or a proliferatory component such as restenosis, stenosis and atherosclerosis; and combinations thereof. Inflammatory or immune-associated diseases or disorders also include, but are not limited to endocrine disorders, rheumatic disorders, collagen diseases, dermatologic disease, allergic disease, ophthalmic disease, respiratory disease, hematologic disease, gastrointestinal disease, inflammatory disease, autoimmune disease, congenital adrenal hyperplasia, nonsuppurative thyroiditis, hypercalcemia associated with cancer, juvenile rheumatoid arthritis, ankylosing spondylitis, acute and subacute bursitis, acute nonspecific tenosynovitis, acute gouty arthritis, post-traumatic osteoarthritis, synovitis of osteoarthritis, epicondylitis, acute rheumatic carditis, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis, iridocyclitis, chorioretinitis, optic neuritis, symptomatic sarcoidosis, fulminating or disseminated pulmonary tuberculosis, idiopathic thrombocytopenic purpura in adults, secondary thrombocytopenia in adults, acquired (autoimmune) hemolytic anemia, leukemias and lymphomas in adults, acute leukemia of childhood, regional enteritis, autoimmune vasculitis, multiple sclerosis, chronic obstructive pulmonary disease, solid organ transplant rejection, sepsis and combinations thereof. Particular treatments include treatment of transplant rejection, rheumatoid arthritis, psoriatic arthritis, multiple sclerosis, Type I diabetes, asthma, IBD, systemic lupus erythematosis, psoriasis and chronic pulmonary disease.

In one aspect the disorder responsive to increased cellular level of one or more anti-inflammatory cytokines such as IL-10 and/or IL-13 is an inflammatory disease or disorder, for example any such disease or disorder listed above.

In some embodiments, a method is provided for treating a disorder related to increased or excessive activity of one or more pro-inflammatory transcription factors and/or increased or excessive production of one or more pro-inflammatory cytokines, comprising administering to a subject having such a disorder a therapeutically effective amount of at least one compound selected from irindalone, physiologically active enantiomers thereof, and pharmaceutically acceptable salts thereof.

A “disorder related to increased or excessive activity of one or more pro-inflammatory transcription factors and/or increased or excessive production of one or more pro-inflammatory cytokines” herein is any disorder associated with or resulting, in whole or in part, from increased or excessive activity of a pro-inflammatory transcription factor such as AP-1, NF-κB or both, and/or increased or excessive production of a pro-inflammatory cytokine such as TNF-α, IL-1α, CXCL1, eotaxin or a combination thereof, in a subject.

Examples of such disorders include, without limitation, any of those listed above as responsive to increased cellular level of anti-inflammatory cytokines.

In some embodiments, a method is provided for treating or preventing an immune disorder in a subject, comprising administering to the subject a therapeutically effective amount of at least one compound selected from irindalone, physiologically active enantiomers thereof, and pharmaceutically acceptable salts thereof. In a more particular embodiment, a method is provided for treating an immune disorder, comprising administering to a subject having such a disorder a therapeutically effective amount of at least one compound selected from irindalone, physiologically active enantiomers thereof, and pharmaceutically acceptable salts thereof.

Examples of immune disorders treatable by such a method include autoimmune diseases such as rheumatoid arthritis, psoriatic arthritis, multiple sclerosis, Type I and Type II diabetes, juvenile diabetes, obesity, asthma, IBD (such as Crohn's disease and ulcerative colitis), pyoderma gangrenum, lupus (systemic lupus erythematosis), myasthenia gravis, psoriasis, dermatitis, dermatomyositis, eczema, seborrhoea, pulmonary inflammation, uveitis, hepatitis, Graves' disease, Hashimoto's thyroiditis, autoimmune thyroiditis, Behcet's or Sjögren's syndrome (dry eyes/mouth), pernicious or immunohemolytic anemia, atherosclerosis, Addison's disease (autoimmune disease of the adrenal glands), idiopathic adrenal insufficiency, autoimmune polyglandular disease (autoimmune polyglandular syndrome), glomerulonephritis, scleroderma, morphea, lichen planus, vitiligo (depigmentation of the skin), alopecia areata, autoimmune alopecia, autoimmune hypopituitarism, Guillain-Barré syndrome, alveolitis and combinations thereof.

The method according to all of the above embodiments comprises administering to the subject having a condition or disorder as specified a therapeutically effective amount of at least one compound selected from irindalone, physiologically active enantiomers thereof, and pharmaceutically acceptable salts thereof.

“Irindalone” and its synonyms Lu 21-098, GL10002 and ORE10002 herein refer specifically to (+)-(1R,3S)-1-[2-]4-[3-(p-fluorophenyl)-1-indanyl]-1-piperazinyl]-ethyl]-2-imidazolidinone, i.e., the compound of formula

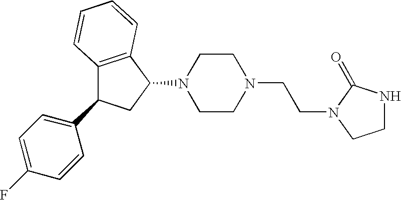

including its solvates, hydrates and polymorphs.

Enantiomers of irindalone include (−)-(1S ,3R)-1-[2-1:4-[3-(p-fluorophenyI)-1-indany]-1-1-piperazinyl]-ethyl]-2-imidazolidinone, and its solvates, hydrates and polymorphs, Mixtures of enantiomers in any proportion, including racemic mixtures, can also be used.

Irindalone and its (−)-(1S,3R)-enantiomer are disclosed in U.S. Pat. No. 4,684,650 to Bogeso, at Example 1 thereof, and can be prepared by the process set forth therein or by any other process known in the art. The disclosure of U.S. Pat. No. 4,684,650 is incorporated herein by reference in its entirety.

In the present context “physiologically active” means having a detectable effect on activity of AP-1 and/or NF-κB, and/or on production of TNF-α, IL-1α, CXCL1, eotaxin, IL-10 and/or IL-13 in a suitable in vitro or in vivo assay, for example as illustrated in the Examples herein. It will be understood that relative affinity of enantiomers for 5-HT_{2A} is not necessarily reflective of relative activity or efficacy for use according to the present method.

In one embodiment, the compound administered is irindalone or a pharmaceutically acceptable salt thereof.

A “solvate” for the purpose of this invention is a solid-state complex of a compound (e.g., irindalone) with a solvent. Exemplary solvates include, but are not limited to, complexes of the compound with ethanol or methanol. A hydrate is a specific form of solvate wherein the solvent is water. Both free base forms of the compound and salts thereof can form solvates and/or hydrates.

The phrase “pharmaceutically acceptable” herein refers to materials, for example salts of irindalone or excipients used in irindalone formulations, which are, within the scope of sound medical judgment, suitable for use in contact with tissues of human subjects without excessive toxicity, irritation, injury or other problem or complication, commensurate with a reasonable benefit/risk ratio.

Irindalone and enantiomers thereof have protonatable nitrogen atoms and therefore typically behave as bases. These compounds react with organic and inorganic acids to form acid addition salts by means well known in the art. Such acid addition salts include, for example, acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxy-ethanesulfonate, itaconate, lactate, maleate, mandelate, methanesulfonate, 2-naphthalene-sulfonate, nicotinate, nitrate, oxalate, pamoate, pectinate, persulfate, 3-phenylpropionate, picrate, pivalate, propionate, succinate, sultanate, tartrate, thiocyanate, tosylate and undecanoate salts and mixtures thereof.

Although a compound selected from irindalone, enantiomers thereof and pharmaceutically acceptable salts thereof can be administered as a compound per se, it will generally be found preferable to administer it as an active ingredient of a pharmaceutical composition. The compound can be the sole active ingredient of such a composition, or it can be accompanied by one or more additional active ingredients. In some embodiments, a method for treating an inflammatory condition comprises administering to a subject having such a condition a composition consisting essentially of a therapeutically effective amount of at least one compound selected from irindalone, physiologically active enantiomers thereof, and pharmaceutically acceptable salts thereof. In some embodiments, a method for treating a disorder responsive to increased cellular level of one or more anti-inflammatory cytokines, such as IL-10 and/or IL-13, comprises administering to a subject having such a disorder a composition consisting essentially of a therapeutically effective amount of at least one compound selected from irindalone, physiologically active enantiomers thereof, and pharmaceutically acceptable salts thereof. In some embodiments, a method for treating a disorder related to excessive activity of one or more pro-inflammatory transcription factors such as AP-1 and/or NF-κB, and/or excessive production of one or more pro-inflammatory cytokines such as TNF-α, IL-1α, CXCL1 or eotaxin, comprises administering to a subject having such a disorder a therapeutically effective amount of at least one compound selected from irindalone, physiologically active enantiomers thereof, and pharmaceutically acceptable salts thereof. In some embodiments, a method for treating or preventing an immune disorder in a subject comprises administering to the subject a therapeutically effective amount of at least one compound selected from irindalone, physiologically active enantiomers thereof, and pharmaceutically acceptable salts thereof.

A pharmaceutical composition useful herein comprises a compound selected from irindalone, enantiomers thereof and pharmaceutically acceptable salts thereof, together with at least one pharmaceutically acceptable excipient. One or more such excipients can serve as a vehicle or carrier for the active ingredient. Choice of vehicle depends on a number of factors, but principally on the route of administration.

Examples of routes of administration which can be used include parenteral, oral, mucosal, ocular, intrapulmonary (e.g., by inhalation), dermal (topical) and transdermal routes, and by implantation.

Parenteral administration, for example by injection or infusion, includes without limitation intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal administration. Parenteral dosage fauns can be adapted to provide immediate release of the active ingredient or can be depot forms providing sustained release over a more prolonged period of time. Parenteral administration generally provides systemic delivery of the active ingredient. Parenteral dosage forms are typically liquid solutions or suspensions and can have an aqueous or non-aqueous (e.g., oily) carrier.

Where it is desired to administer the active ingredient parenterally or intravascularly to a local area in need of treatment, this can be achieved, for example, by local infusion during surgery, by injection, by means of a catheter, or by means of an implant having the active ingredient in or on a porous, non-porous or gelatinous material, including a membrane (e.g., a silastic membrane), fiber or wafer (e.g., a polifeprosan 20 wafer).

Oral administration (i.e., administration per os or p.o.) can be in the form of a liquid formulation such as a solution, syrup or suspension, or a solid dosage form such as a tablet or capsule. Such dosage forms can be adapted for immediate or controlled (e.g., sustained or delayed) release. Oral administration generally provides systemic delivery of the active ingredient.

Mucosal (i.e., transmucosal) administration can occur via any mucosal tissue, including without limitation oral (e.g., sublingual or buccal), nasal (intranasal), rectal and vaginal mucosa. Dosage forms suitable for treating mucosal tissues within the oral cavity can be formulated as mouthwashes or as oral gels. Mucosal administration can be adapted for systemic or local delivery.

Ocular administration can provide systemic delivery, but is especially well adapted to local delivery, for example to treat an inflammatory condition of the eye such as uveitis.

In some embodiments, the pharmaceutical composition is administered topically to skin (“dermal” administration providing local delivery to skin tissues, as distinct from “transdermal” administration, which is a form of systemic delivery through the skin). Topical administration to skin is especially useful where the condition to be treated comprises inflammation of the skin, as for example in psoriasis.

While it can be possible to administer irindalone or an enantiomer thereof or a salt thereof unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Processes for preparing pharmaceutical compositions include bringing into association the active ingredient with a diluent and, optionally, one or more accessory ingredients, to form the pharmaceutical composition or dosage form. Optional accessory ingredients include such excipients as preservatives, wetting agents, emulsifying agents, dispersing agents, emollients, etc. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, lotions, liposome preparations, nanoparticulate preparations, injectable preparations, enemas, suppositories, inhalable powders, sprayable liquids, aerosols, patches, depots and implants.

Illustratively, in a liquid formulation suitable, for example, for parenteral, intranasal, intrapulmonary (for example as an aerosol) or oral delivery, the API can be present in solution or suspension, or in some other form of dispersion, in a liquid medium that comprises a diluent such as water. Additional excipients that can optionally be present in such a formulation include a tonicifying agent, a buffer (e.g., a tris, phosphate, imidazole or bicarbonate buffer), a dispersing or suspending agent and/or a preservative. Such a formulation can contain micro- or nanoparticulates, micelles and/or liposomes. Excipients suitable for use in liquid formulations include, but are not limited to, water, glycols, oils, alcohols, flavoring agents, preservatives and coloring agents. A parenteral formulation can be prepared in dry reconstitutable form, requiring addition of a liquid carrier such as water or saline prior to administration by injection.

Because their administration typically bypasses patients' natural defenses against contaminants, parenteral and intravascular dosage forms are preferably sterile or capable of being sterilized prior to administration to a patient. Examples of parenteral dosage fauns include, but are not limited to, solutions ready for injection, dry products (including, but not limited to, lyophilized powders, pellets and tablets) ready to be dissolved or suspended in a pharmaceutically acceptable vehicle for injection, suspensions ready for injection, and emulsions.

Suitable vehicles that can be used to provide parenteral dosage faults of the invention are well known to those skilled in the art. Examples include, but are not limited to: water for injection USP; aqueous vehicles such as, but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of an active ingredient can also be incorporated into a parenteral dosage form; examples of such excipients include cyclodextrins.

For rectal delivery, the API can be present in dispersed form in a suitable liquid (e.g., as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents or fillers illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), low moisture starches (e.g., Starch 1500 LM), starch components such as amylose and dextrates, and modified or processed starches such as pregelatinized starch; dextrins; celluloses including powdered cellulose, microcrystalline cellulose, silicified microcrystalline cellulose, food grade sources of a- and amorphous cellulose and powdered cellulose, and cellulose acetate; calcium salts including calcium carbonate (e.g., in granular or powder form), tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. In some embodiments one or more diluents are present in a total amount of about 50% to about 99% by weight of the composition. The diluent or diluents selected preferably exhibit suitable flow properties and, where tablets are desired, compressibility.

Lactose, microcrystalline cellulose and starch, either individually or in combination, are particularly useful diluents. Suitable forms of microcrystalline cellulose include, but are not limited to, materials sold as Avicel™ PH-101, Avicel™ PH-103 and Avicel™ PH-105 by FMC Corp., Americal Viscose Division, Marcus Hook, Pa., and products equivalent thereto. A mixture of microcrystalline cellulose and carmellose sodium is sold, for example, as Avicel™ RC-581.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth (e.g., powdered tragacanth); glucose; polydextrose; starch including pregelatinized starch; gelatin; modified celluloses including cellulose acetate, methylcellulose, carmellose calcium, carmellose sodium, hydroxypropylmethylcellulose (HPMC or hypromellose, illustratively types 2208, 2906 and 2910), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginic acid and alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition. In some embodiments one or more disintegrants are present in a total amount of about 0.5% to about 15%, more specifically about 1% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lautyl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium, zinc and sodium stearates; mineral oil including light mineral oil, hydrogenated vegetable oils (e.g., peanut, cottonseed, sunflower, sesame, olive, corn and soybean oils); glycerin, glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; ethyl laurate; ethyl oleate; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. In some embodiments one or more lubricants are present in a total amount of less than about 1% by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide (e.g., Aerosil™ 200, Cab-O-Sil™ and products equivalent thereto), starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individunlly or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. A tablet can be prepared by compression or molding. Compressed tablets can be prepared by compressing in a suitable machine the active ingredients in a free-flowing form such as powder or granules, optionally mixed with an excipient. Tablets can alternatively be made by molding in a suitable machine a mixture of the powdered compound moistened with an inert liquid. Capsules can have hard or soft shells comprising, for example, gelatin and/or HPMC, optionally together with one or more plasticizers.

A pharmaceutical composition useful herein typically contains the active ingredient in an amount of about 1% to about 99%, more typically about 5% to about 90% or about 10% to about 60%, by weight of the composition. A unit dosage form such as a tablet or capsule can conveniently contain an amount of the compound providing a single dose, although where the dose required is large it may be necessary or desirable to administer a plurality of dosage forms as a single dose. Illustratively, a unit dosage form can comprise the active ingredient in an amount of about 0.01 mg to about 1,000 mg free base equivalent, more typically about 0.1 mg to about 250 mg, for example about 0.5 mg to about 100 mg or about 1 mg to about 50 mg, illustratively about 1 mg to about 5 mg.

Typical excipients for transdermal, topical and mucosal dosage forms include, but are not limited to, water, acetone, ethanol, ethylene glycol, propylene glycol, 1,3-butanediol, isopropyl myristate, isopropyl palmitate, mineral oil and mixtures thereof to form lotions, tinctures, creams, emulsions, gels or ointments. Moisturizers or humectants can also be added if desired.

In compositions for topical application to skin, the carrier typically comprises a pharmaceutically acceptable solvent for the active ingredient. Where the active ingredient is in a water-soluble form such as a water-soluble salt, water is a suitable solvent. For an active ingredient of low water solubility, one or more pharmaceutically acceptable organic solvents can be used.

As an oily base for an ointment formulation, a hydrocarbon base such as petrolatum, with optional admixture of one or more waxes, can be used. The ointment can be made more hydrophilic by addition of solvents such as those listed above, illustratively cholesterol, stearyl alcohol and/or PEG. A topical preparation wherein the vehicle is a semi-solid emulsion having a hydrophobic (e.g., petrolatum) phase and a hydrophilic (e.g., PEG) phase, stabilized with one or more emulsifying agents (e.g., sodium lauryl sulfate) is generally known as a cream. A semi-solid water-based topical preparation having a hydrophilic gelling or thickening agent and no hydrophobic phase is generally known as a gel. Where the topical preparation is liquid rather than semi-solid, it is generally known as a lotion. Lotions can be simple solutions, suspensions or emulsions, including microemulsions.

Among suitable organic solvents for topical use are mono-, di- and polyhydric alcohols, illustratively including ethanol, isopropanol, n-butanol, 1,3-butanediol, propylene glycol, glycerol, glycofurol, cholesterol, myristyl alcohol, oleyl alcohol, stearyl alcohol and polyethylene glycol (PEG), e.g., PEG having an average molecular weight of about 200 to about 800. Suitable PEGs include PEG-200, PEG-350, PEG-400, PEG-540 and PEG-600. Some of the above solvents can function additionally as skin permeation enhancers.

Alternatively or in addition, a pharmaceutically acceptable glycol ether solvent can be used. Glycol ethers useful herein typically have a molecular weight of about 75 to about 1000, for example about 75 to about 500 or about 100 to about 300.

Non-limiting examples of glycols and glycol ethers useful herein include ethylene glycol monomethyl ether, ethylene glycol dimethyl ether, ethylene glycol monoethyl ether, ethylene glycol diethyl ether, ethylene glycol monobutyl ether, ethylene glycol dibutyl ether, ethylene glycol monophenyl ether, ethylene glycol monobenzyl ether, ethylene glycol butylphenyl ether, ethylene glycol terpinyl ether, diethylene glycol monomethyl ether, diethylene glycol dimethyl ether, diethylene glycol monoethyl ether, diethylene glycol diethyl ether, diethylene glycol divinyl ether, ethylene glycol monobutyl ether, diethylene glycol dibutyl ether, diethylene glycol monoisobutyl ether, triethylene glycol dimethyl ether, triethylene glycol monoethyl ether, triethylene glycol monobutyl ether, tetraethylene glycol dimethyl ether, and mixtures thereof. See for example Flick (1998) Industrial Solvents Handbook, 5th ed., Noyes Data Corporation, Westwood, N.J.

An illustratively suitable glycol ether solvent is diethylene glycol monoethyl ether, sometimes referred to in the art as DGME or ethoxydiglycol. It is available for example under the trademark Transcutol™ of Gattefossé Corporation.

Compositions for topical administration optionally comprise one or more pharmaceutically acceptable co-solvents. Non-limiting examples of co-solvents suitable herein include any solvent listed above; N-methyl-2-pyrrolidinone (NMP); oleic and linoleic acid triglycerides, for example soybean oil; caprylic/capric triglycerides, for example Miglyol™ 812 of Huls; caprylic/capric mono- and diglycerides, for example Capmul™ MCM of Abitec; benzyl phenylformate; diethyl phthalate; triacetin; polyoxyethylene caprylic/capric glycerides such as polyoxyethylene (8) caprylic/capric mono- and diglycerides, for example Labrasol™ of Gattefossé; medium chain triglycerides; propylene glycol fatty acid esters, for example propylene glycol laurate; oils, for example corn oil, mineral oil, cottonseed oil, peanut oil, sesame seed oil and polyoxyethylene (35) castor oil, for example Cremophor™ EL of BASF; polyoxyethylene glyceryl trioleate, for example Tagat™ TO of Goldschmidt; polyoxyethylene sorbitan esters, for example polysorbate 80; and lower alkyl esters of fatty acids, for example ethyl butyrate, ethyl caprylate and ethyl oleate.

Another optional component of the carrier is a skin permeation enhancer.

In one embodiment, a permeation enhancer selected from terpenes, terpenoids, fatty alcohols and derivatives thereof is present in the carrier. Examples include oleyl alcohol, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof. Optionally more than one such permeation enhancer, for example a fatty alcohol and a terpene or terpenoid, can be present. Thus, in an illustrative embodiment, a topical composition of the invention comprises as penetration enhancers oleyl alcohol and thymol.

Fatty acids such as oleic acid and their alkyl and glyceryl esters such as isopropyl laurate, isopropyl myristate, methyl oleate, glyceryl monolaurate, glyceryl monooleate, glyceryl dilaurate, glyceryl dioleate, etc. also can be used as permeation enhancers. Fatty acid esters of glycolic acid and its salts, for example as disclosed in International Patent Publication No. WO 98/18416, incorporated herein by reference, are also useful permeation enhancers. Examples of such esters include lauroyl glycolate, caproyl glycolate, cocoyl glycolate, isostearoyl glycolate, sodium lauroyl glycolate, tromethamine lauroyl glycolate, etc. Also useful as permeation enhancers are lactate esters of fatty alcohols, for example lauryl lactate, myristyl lactate, oleyl lactate, etc.

Other permeation enhancers include hexahydro-1-dodecyl-2H-1-azepin-2-one (laurocapram, Azone™) and derivatives thereof, acetone, alkyl sulfoxides, e.g., dimethylsulfoxide (DMSO) and n-decyl methylsulfoxide, salicylic acid and alkyl esters thereof, e.g., methyl salicylate, tetrahydrofuryl alcohol, urea, N,N-dimethylacetamide, dimethylformamide, N,N-dimethyltoluamide, 2-pyrrolidinone and N-alkyl derivatives thereof, e.g., NMP and N-octyl-2-pyrrolidinone, 2-nonyl-1,3-dioxolane, eucalyptol and sorbitan esters.

In a particular embodiment, the carrier comprises as a permeation enhancer a sunscreen. This can be an ester sunscreen as described, for example, in International Patent Publication No. WO 97/29735, incorporated herein by reference. Examples include alkyl esters of p-aminobenzoic acid (PABA), p-dimethylaminobenzoic acid, 2-amimobenzoic acid, cinnamic acid, p-methoxycinnamic acid, salicylic acid and 2-cyano-3,3-diphenylacrylic acid, for example 2-ethylhexyl p-dimethylaminobenzoate (Padimate O), 2-ethylhexyl p-methoxy-cinnamate, 2-ethylhexyl salicylate, menthyl salicylate, homomenthyl salicylate (homosalate), menthyl 2-aminobenzoate and 2-ethylhexyl 2-cyano-3,3-diphenylacrylate (octocrylene). Usefulness of such compounds as permeation enhancers is not necessarily correlated with their effectiveness as sunscreens.

Alternatively the sunscreen can be other than an ester sunscreen, for example a benzophenone sunscreen or modification thereof, such as 2-hydroxy-4-methoxybenzophenone (oxybenzone), 2,2′-dihydroxy-4-methoxybenzophenone (dioxybenzone), 5-benzoyl-4-hydroxy-2-methoxybenzenesulfonic acid (sulisobenzone) or 1-(p-tert-butylphenyl)-3-(p-methoxyphenyl)-1,3-propanedione (avobenzone). Optionally other typical ingredients of sunscreen preparations can be included, such as titanium dioxide.

Other ingredients of the carrier can include one or more excipients selected from thickening agents, surfactants, emulsifiers, antioxidants, preservatives, stabilizers, colors and fragrances. A skin irritation reducing agent, such as vitamin E, glycyrrhetic acid or diphenhydramine, can also be present.

Any liquid or semi-solid dosage form suitable for topical application to skin can be useful herein and can be formulated according to conventional methods known in the art. A dosage form as contemplated herein can be non-occlusive or occlusive, i.e., having a backing material. Suitable dosage forms for topical use include a cream, paste, gel, ointment, lotion, sprayable liquid (e.g., aerosol), plaster or patch of the matrix or reservoir type.

A non-limiting illustrative paste, ointment, gel or cream is a composition of the invention comprising irindalone or a physiologically active enantiomer thereof or a pharmaceutically acceptable salt thereof, at least one solvent, at least one skin permeation enhancer and at least one thickening agent. Suitable thickening agents for ointments, gels and creams include without limitation hydroxypropylcellulose, hydroxypropylmethylcellulose (HPMC), hydroxyethylcellulose, ethylcellulose, carboxymethylcellulose, dextran, guar gum, polyvinylpyrrolidone (PVP or povidone), pectin, starch, gelatin, casein, acrylic acid, acrylic acid esters, acrylic acid copolymers, vinyl alcohols, alkoxy polymers, polyethylene oxide polymers, polyethers and the like.

An embodiment of the invention is a composition suitable for application to skin by means of an applicator such as an aerosol, a spray, a pump-pack, a brush or a swab. Preferably, such an applicator provides fixed or variable metered dose application, as exemplified by a metered dose aerosol, a stored-energy metered dose pump or a manual metered dose pump. According to this embodiment, application is most preferably performed by means of a topical metered-dose aerosol combined with an actuator nozzle shroud which together accurately control the amount and/or uniformity of the dose applied. The shroud can help control the distance of the nozzle from the skin, a function that can alternatively be achieved by means of a spacer-bar or the like. Another function of the shroud is to enclose the treated area of the skin in order to prevent or limit bounce-back and/or loss of the composition. Typically the area of application defined by the shroud is substantially circular in shape. The composition may be propelled by a pump-pack or by use of an aerosol propellant such as a hydrocarbon or hydrofluorocarbon propellant, nitrogen, nitrous oxide, carbon dioxide or an ether, for example dimethyl ether.

The active ingredient in a topical formulation can, in some embodiments, be encapsulated, for example in microcapsules or liposomes.

Topical applications typically contain a relatively low concentration of the active ingredient, for example about 0.01% to about 10% by weight, wherein the active ingredient, even if administered in the form of a salt, is expressed as free base equivalent. More typically, the active ingredient concentration in a topical formulation useful herein is about 0.1% to about 10% by weight.

Suitable doses of irindalone or a physiologically active enantiomer thereof, or a pharmaceutically acceptable salt thereof, providing a therapeutically effective amount will vary, depending, for example, on age and body weight of the subject, whether single or multiple administrations are given, route of administration, the particular condition or disorder to be treated, severity of the condition or disorder, the desired objective (e.g., stabilization or slowing progression of the condition or disorder, alleviation of pain associated with inflammation, prevention of flares of the disorder, etc.), use in monotherapy or combination therapy (for example with another anti-inflammatory drug or with an agent addressing an underlying or complicating disease), tolerance of the individual for potential side-effects such as hypotension, and other factors known to those of skill in the art. The physician of ordinary skill can, without undue experimentation, develop a dosage regimen appropriate for any specific situation to provide an optimum therapeutic response tailored to an individual patient. For example, the physician can titrate the dose upward to increase efficacy or downward to reduce any undesirable side-effect that may occur at higher doses.

A “therapeutically effective amount” herein is an amount that is sufficient to palliate, ameliorate, stabilize, reverse or slow the progression of an inflammatory condition or other disorder as disclosed herein, or the symptoms thereof. It is not generally contemplated that irindalone or an enantiomer thereof will remove an underlying cause of the inflammatory or other condition and thereby effect a permanent “cure” for the condition. A therapeutically effective amount can be provided in one or a series of administrations. Therapeutically effective doses are expressed herein on a per diem or “daily dose” basis without implication that the compound or composition is necessarily administered once daily. Suitable dosage intervals can range from about 2 hours to about 30 days (or even longer in the case of implants), depending on release properties of the composition and other factors, but will more typically be in the range of about 8 hours to about 7 days, providing an administration frequency of about three times a day to about once a week.

The daily dose of irindalone or enantiomer thereof or salt thereof in practice of the present invention is generally about 0.01 mg to about 1,000 mg free base equivalent, more typically about 0.1 mg to about 250 mg, for example about 0.5 mg to about 100 mg or about 1 mg to about 50 mg, illustratively about 1 mg to about 5 mg. Expressed on the basis of body weight, suitable daily doses are generally about 0.001 to 25 mg/kg.

In those embodiments of the invention wherein a cell is contacted with a compound selected from irindalone, enantiomers thereof and salts thereof, such contacting can be in vivo or, in certain embodiments, in vitro. Contacting a cell in vivo can include administration of a composition comprising the compound to a subject, or to a tissue of a subject, such that a cell is contacted with the compound. Contacting a cell in vitro can include, e.g., contacting the cell with the compound per se, or with a composition comprising or consisting essentially of the compound, directly or by addition of compound or composition to a growth medium for the cell.

This invention also encompasses kits that, when used by a medical practitioner, can simplify identification of subjects and administration of appropriate amounts of irindalone or an enantiomer thereof or a pharmaceutically acceptable salt thereof to a patient.

An illustrative kit of the invention comprises one or more unit dosage forms of irindalone or an enantiomer thereof or a pharmaceutically acceptable salt thereof, and instructions for identification of a subject.

Kits of the invention can further comprise devices that are used to administer the irindalone or enantiomer thereof or salt thereof. Examples of such devices include, but are not limited to, intravenous cannulation devices, syringes, drip bags, patches, topical gels, pumps, containers that provide protection from photodegradation, autoinjectors and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to water for injection USP; aqueous vehicles such as, but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol and polypropylene glycol; and non-aqueous vehicles such as, but not limited to, corn oil, cottonseed oil, peanut oil, sesame oil, ethyl oleate, isopropyl myristate and benzyl benzoate.

In this disclosure, “comprises,” “comprising,” “containing” and the like have the meaning ascribed to them in U.S. patent law and can mean “includes,” “including” and the like; “consisting essentially of” and “consists essentially of” likewise have the meaning ascribed to them in U.S. patent law and are open-ended, allowing for presence of more than that which is expressly recited so long as basic or novel characteristics of that which is recited is not changed by such presence, but excluding prior art embodiments.

Those skilled in the art will recognize, or be able to ascertain using no more than routine experimentation, equivalents of the specific embodiments of the invention described herein.

Prior publications, patent applications and patents cited herein are incorporated by reference.

### Examples

In order that the invention may be more fully understood, the following examples are provided. These examples are for illustrative purposes only and are not to be construed as limiting the invention in any way. Irindalone was used in the examples in the form of irindalone tartrate, and amounts and doses are expressed as irindalone tartrate.

### Example 1

### Irindalone Has a Bi-Phasic Effect on LPS-Induced NF-κB:Luciferase Reporter Activation

Effects of irindalone on rodent physiological processes were evaluated using bio-photonic in vivo imaging. Specifically, this technology was used to investigate temporal and spatial modulation of physiological processes following acute compound administration. Transgenic mice expressing a firefly luciferase reporter driven by a series of promoters that represent a wide spectrum of potential disease states were used. Light emitted by luciferase in the presence of a luciferin substrate was detected and analyzed using a highly sensitive CCD imaging system. Mice were anesthetized and imaged at the indicated time points followed by visual and quantitative analysis based on counting photons of light emitted from specific regions of interest. Observations were followed by confirmatory and additional heuristic studies, including direct measurement of luciferase activity in harvested tissues and organs.

As detailed below, the properties of irindalone were analyzed on three distinct transgenic lines, NF-κB:LUC, AP-1:LUC and HO-1:LUC. For this Example, the NF-κB transgenic mouse line was constructed using three NF-κB response elements fused to a firefly luciferase reporter. The effect of acute compound administration on inflammatory and immunomodulatory processes was monitored using activation of the luciferase reporter as a surrogate readout for activation of NF-κB transcription factor.

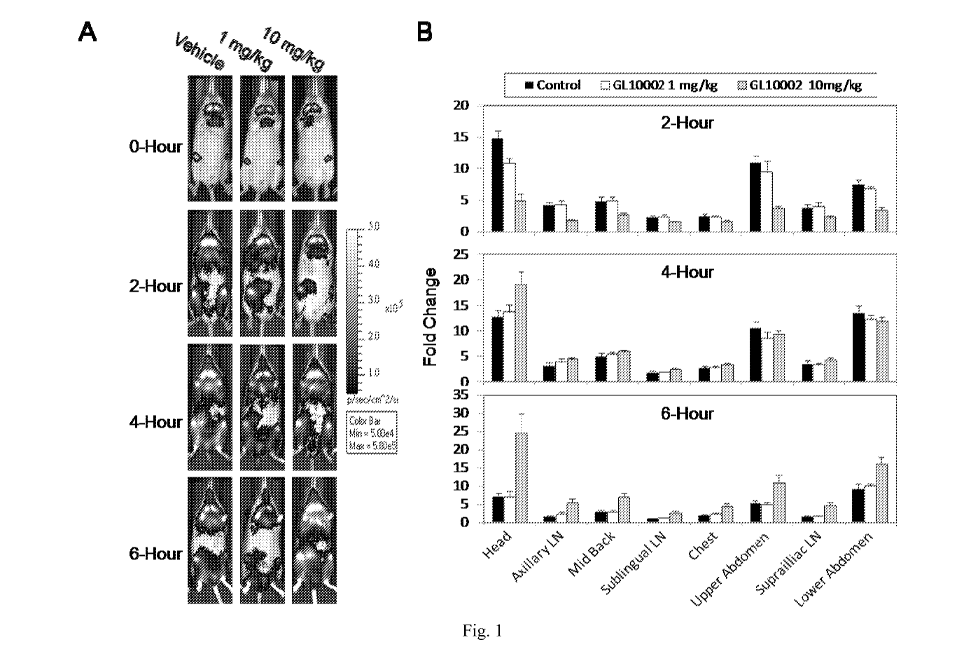

As can be seen in FIG. 1, administration of irindalone resulted in a bi-phasic effect on NF-κB activation. For these studies, male mice (n=5 per group) were administered irindalone (0, 1 or 10 mg/kg, p.o.) one hour prior to injection with soluble lipopolysaccharide (sLPS, 2.5 mg/kg, i.v.). Whole body images were obtained prior to drug pretreatment (0 hour time point) and again at 2, 4, and 6 hours post-LPS treatment. Data (mean±SEM) are expressed as “fold change” from 0 hour, i.e., as a multiple of the activation level measured at 0 hour. hindalone exhibited broad suppression of LPS-induced NF-κB reporter after 2 hours and broad enhancement of LPS-induced NF-κB reporter after 6 hours.

### Example 2

### Irindalone Exhibits Effects on Quantities of Circulating Plasma Cytokines

The intriguing bi-phasic modulation of NF-κB activation described in Example 1 was further investigated by repeating the LPS induction experiment and performing subsequent imaging analysis at 0 hour and 2, 4, 6 and 24 hours post-LPS treatment. Moreover, blood samples were collected at each of the timepoints and plasma was prepared for evaluation of effects of irindalone on quantities of circulating cytokines.

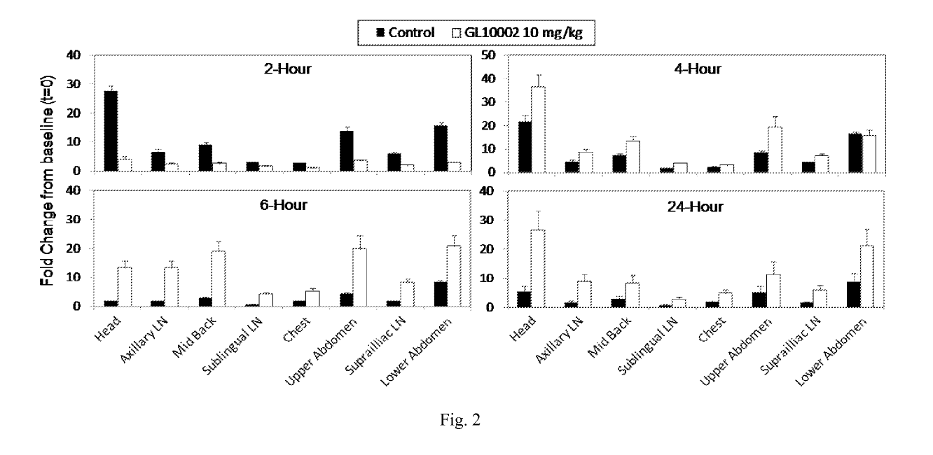

As shown in FIG. 2, image analysis detected a suppression of LPS-induced NF-κB activation at the early time point (2 hours) with an enhancement in NF-κB activation at later time points. Subsequent plasma cytokine analysis via bead-based immunoassay (Luminex Corp.) demonstrated that irindalone had a broad effect on circulating cytokines (FIG. 3). Specifically, irindalone suppressed LPS-induced pro-inflammatory cytokines eotaxin (FIG. 3A) and TNF-α (FIG. 3B) at the 2-hour time point and IL-1α at both 2 hours and 6 hours post-LPS injection (FIG. 3C). Moreover, irindalone was found to increase the anti-inflammatory cytokine IL-10 at all time points measured, with the most pronounced effect at 2 and 4 hours (FIG. 3D).

### Example 3

### Irindalone Exhibits a Tissue-Selective Effect on LPS-Induced NF-κB Activation with the Most Pronounced Changes Observed in Visceral Fat, Gastrointestinal Tract and Brain Tissues

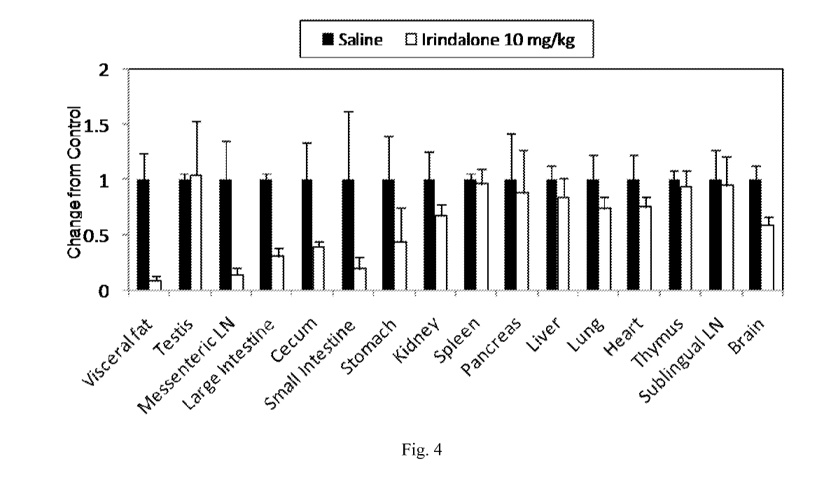

The above described results highlighting the effects of irindalone on both LPS-induced NF-κB reporter activity and plasma cytokine levels indicate that irindalone possesses immune-modulatory activity. To determine whether these effects can be localized to a particular tissue or organ system, additional experimentation was performed (similar in design to that as described in Example 1). For these studies, male mice (n=5 per group) were administered irindalone (0 and 10 mg/kg, p.o.) one hour prior to injection with LPS (2.5 mg/kg, i.v.). Whole body images were obtained prior to drug pretreatment (0 hour) and again at 2 hours post-LPS treatment (data not shown). A broad collection of internal tissues and organs were then removed and snap frozen for further analysis. Specifically, harvested tissues were homogenized, cells were lysed and utilized for luciferase and protein quantification assays to obtain relative units of light/μg protein. Results (FIG. 4) illustrate normalized comparisons between LPS alone versus irindalone+LPS.

### Example 4

### Irindalone Can Suppress TPA-Induced Activation of AP-1:LUC Reporter Activity in Skin

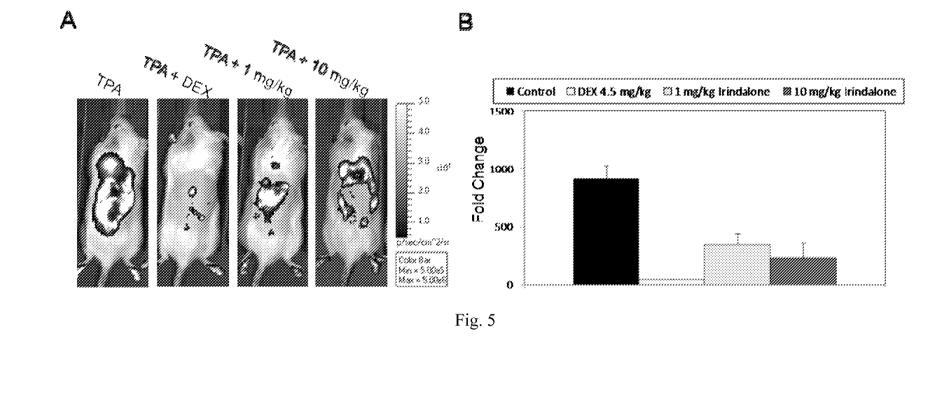

The AP-1:LUC transgenic line was constructed with four copies of the AP-1 response element fused to a firefly luciferase reporter. Activation of the Fos and Jun heterodimer transcription factor (AP-1) via MAP kinase signaling can modulate luciferase reporter expression. AP-1 activation has been linked to both inflammatory and proliferative responses. For the current studies, the backs of mice (n=5 per group) were shaved (area size approximately 1.5×3 cm) and pre-imaged. Mice were administered vehicle (saline), irindalone (dosing at 1 and 10 mg/kg; p.o.) or dexamethasone as a positive control (DEX, 4.5 mg/kg, i.p.) one hour prior to topical application of the phorbol ester 12-O-tetradecanoyl-phorbol-13-acetate (TPA, 0.05 mg/ml in acetone; total dose 10 μg). As shown in FIG. 5, TPA induced AP-1 activity in control animals and irindalone dramatically repressed the TPA-induced AP-1 activation in a dose-dependent manner. The effect was qualitatively similar to that observed with the positive control compound dexamethasone.

### Example 5

### Irindalone Can Suppress Cadmium Chloride Activation of the HO-1:LUC Promoter

The HO-1 luciferase transgenic mouse line was constructed using 15,000 base pairs of the HO-1 promoter fused to a firefly luciferase reporter. The HO-1 promoter is highly responsive to oxidative stress, hypoxia and other stimuli that influence the oxidative state of the animal. Increased expression of the HO-1 pathway limits tissue damage in response to a wide variety of proinflammatory stimuli associated with oxidative stress including hypoxia, hyperoxia, cytokines, nitric oxide (NO), heavy metals, ultraviolet-A radiation, heat shock, shear stress, hydrogen peroxide, and thiol (—SH)-reactive substances, endotoxin, allograft rejection, etc. This promoter region has several enhancer sequences that confer responsiveness to the wide set of stimuli mentioned above. In this mouse model, luciferase expression is an indicator of the induction of the HO-1 promoter/gene expression.

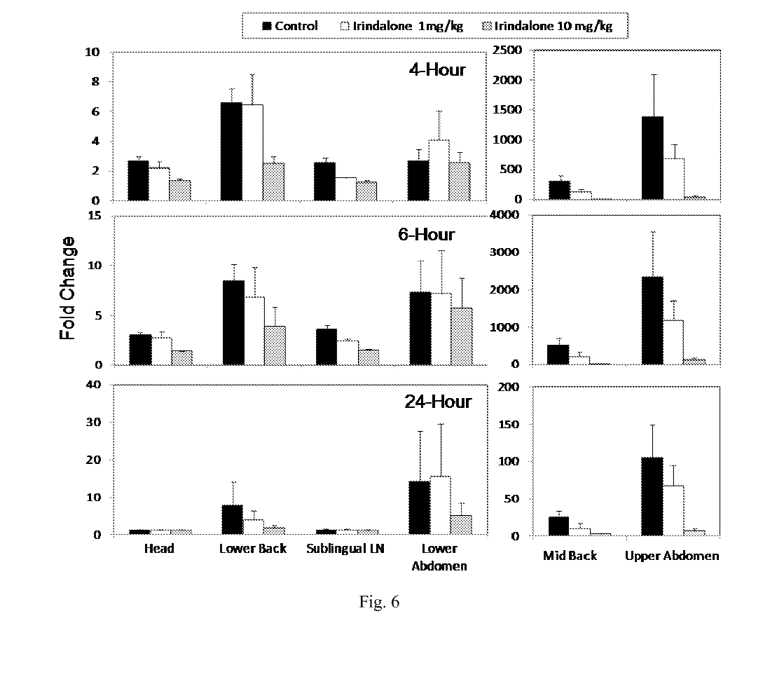

In order to assess the ability of irindalone to modulate the oxidative state of mice stressed with cadmium chloride (CdCl₂), male mice (n=5 per group) were dosed with irindalone (0, 1 or 10 mg/kg, p.o.) one hour prior to injection with CdCl₂ (2 mg/kg, i.v.). Whole body images were obtained prior to irindalone pretreatment (0 hour) and again at 4, 6, and 24 hours post-CdCl₂ treatment. Irindalone suppressed CdCl₂-induced luciferase expression (FIG. 6). Dramatic suppression at all time points was seen in the mid back region and in the sublingual and lower back regions at earlier time points. Additional anatomical regions also posted trends in suppression at early time points.

### Example 6

### Irindalone Increases IL-13 in Rat Plasma and May Modulate CXCL1

The effects of irindalone were also investigated in normal unstimulated rats that were subject to twice-daily oral dosing with irindalone (1 and 5 mg/kg) for 7 consecutive days. Blood was collected approximately 4 hours after administration of the last dose, plasma was prepared and cytokines were measured using a bead-based multiplex immunoassay (Luminex Corp.). As shown in FIG. 7, IL-13 levels were increased in animals that received irindalone compared to those that received vehicle alone, while irindalone also lowered levels of the pro-inflammatory cytokine CXCL1 (GRO/KC), though not dose-proportionately.

What is claimed is:

1. A method for treating an inflammatory condition, comprising administering to a subject having said condition a therapeutically effective amount of at least one compound selected from irindalone, physiologically active enantiomers thereof, and pharmaceutically acceptable salts thereof.

2. The method of claim 1, wherein the at least one compound is administered as an active ingredient of a pharmaceutical composition that comprises the at least one compound and at least one excipient.

3. The method of claim 2, wherein the pharmaceutical composition comprising the at least one compound is adapted for parenteral, oral, mucosal, ocular, intrapulmonary, dermal or transdermal administration or implantation.

4. The method of claim 2, wherein the pharmaceutical composition comprising the at least one compound is administered topically to skin in a form of a cream, ointment, lotion, gel, spray, plaster or patch.

5. The method of claim 1, wherein the at least one compound comprises irindalone or a pharmaceutically acceptable salt thereof.

6. The method of claim 1, wherein the inflammatory condition comprises inflammation of the skin.

7. The method of claim 6, wherein the inflammatory condition comprises psoriasis, eczema, rosacea, acne, burns, dermatitis and/or ultraviolet radiation damage.

8. The method of claim 1, wherein the inflammatory condition comprises inflammatory bowel disease, sepsis, arthritis or multiple sclerosis.

9. A method for treating a disorder responsive to increased cellular level of one or more anti-inflammatory cytokines, comprising administering to a subject having said disorder a therapeutically effective amount of at least one compound selected from irindalone, physiologically active enantiomers thereof, and pharmaceutically acceptable salts thereof.

10. The method of claim 9, wherein the disorder is responsive to increased cellular level of IL-10 and/or IL-13.

11. A method for treating a disorder related to increased or excessive activity of pro-inflammatory transcription factors and/or increased or excessive production of pro-inflammatory cytokines, comprising administering to a subject having said disorder a therapeutically effective amount of at least one compound selected from irindalone, physiologically active enantiomers thereof, and pharmaceutically acceptable salts thereof.

12. The method of claim 11, wherein the disorder is related to increased or excessive activity of AP-1 and/or NF-κB and/or increased or excessive production of IL-1α, CXCL1 and/or eotaxin.

13. A method for elevating level of at least one cytokine selected from the group consisting of IL-10 and IL-13 in a cell, comprising contacting the cell with at least one compound selected from irindalone, physiologically active enantiomers thereof, and pharmaceutically acceptable salts thereof, in an amount effective to elevate level of the at least one cytokine.

14. A method for inhibiting production of at least one cytokine selected from the group consisting of TNF-α, IL-1α, CXCL1 and eotaxin in a cell, comprising contacting the cell with at least one compound selected from irindalone, physiologically active enantiomers thereof, and pharmaceutically acceptable salts thereof, in an amount effective to inhibit production of the at least one cytokine.

15. A method for treating or preventing an immune disorder in a subject, comprising administering to the subject a therapeutically effective amount of at least one compound selected from irindalone, physiologically active enantiomers thereof, and pharmaceutically acceptable salts thereof.

16. A pharmaceutical composition comprising a therapeutically effective amount of at least one compound selected from irindalone, physiologically active enantiomers thereof, and pharmaceutically acceptable salts thereof, in a vehicle comprising at least one pharmaceutically acceptable excipient; said vehicle being adapted for topical administration to skin of a subject.

17. The composition of claim 16, wherein the at least one compound comprises irindalone or a pharmaceutically acceptable salt thereof.

18. The composition of claim 16, wherein the composition is a cream, ointment, gel, lotion, spray, plaster or patch.

19. The composition of claim 16, wherein the amount of the at least one compound is therapeutically effective for treatment of psoriasis.
